(19) United States
(12) Patent Application Publication
(10) Pub. No.: US 2018/0369142 A1
(43) Pub. Date: Dec. 27, 2018

(54) AMPHIPHILIC POLYMER MICELLES AND USES THEREOF

(71) Applicant: NORTHEASTERN UNIVERSITY, Boston, MA (US)

(72) Inventors: Ke ZHANG, Boston, MA (US); Dali WANG, Boston, MA (US)

(21) Appl. No.: 16/011,863

(22) Filed: Jun. 19, 2018

Related U.S. Application Data
(60) Provisional application No. 62/524,405, filed on Jun. 23, 2017.
(60) Provisional application No. 62/525,175, filed on Jun. 26, 2017.

(51) Int. Cl. A61K 9/107; B01J 13/10; C12N 15/113

(52) U.S. Cl. CPC A61K 9/107; B01J 13/10; C12N 2310/14; C12N 2320/32; C12N 2310/11; C12N 15/113

(57) ABSTRACT

By adjusting the spacing and length of particle surface moieties on amphiphilic micelles, hybridization kinetics, nuclease resistance, cellular uptake, and antisense efficacy of amphiphilic micelles are fine tuned.

### CROSS REFERENCE TO RELATED APPLICATIONS

This application claims the benefit of priority under 35 U.S.C. § 119 to U.S. Provisional Patent Application No. 62/524,405 by Zhang and Wang, filed Jun. 23, 2017, and U.S. Provisional Patent Application No. 62/525,175 by Zhang and Wang, filed Jun. 26, 2017, the entire disclosures of which are incorporated herein by reference.

### GOVERNMENT SUPPORT

This invention was made with Government support under Grant No. 1453255 awarded by the National Science Foundation. The Government has certain rights in the invention.

### FIELD OF THE INVENTION

This application relates to micellar nanoparticles. More particularly, this application relates to nucleic acid-, peptide- or small molecule-loaded micellar nanoparticles that are co-assembled from amphiphilic polymers or copolymers and whose surface moieties are adjusted to provide a desired density to the particles.

### BACKGROUND OF THE INVENTION

The use of micelles-based drug delivery has expanded in recent years. Of particular interest is the use of micelles to deliver high concentrations of cytotoxic drugs to diseased tissues selectively, thus reducing the agent's side effects on the rest of the body and the use of micelles to deliver oligonucleotides for anti-sense therapies, for example.

Oligonucleotides (ONs) are considered a form of informational drug, where drug-like properties (pharmacophore) and target information (dianophore) are independent of each other. ON therapeutics promise to dramatically reduce the cost of new drug development, as a change in disease target in principle only requires a change in the ON sequence. However, direct utilization of ONs as a drug is hampered by enzymatic degradation, poor cellular uptake, rapid liver clearance, unwanted activation of the immune system, and overall low biochemical efficacy. Various chemical modifications of the ON or vectors (of viral, polycationic, and liposomal formulations) have been utilized to improve the bioavailability of the nucleic acid. Despite exhaustive efforts, however, these strategies remain subject to several long-standing drawbacks including toxicity, immunogenicity, ON instability, and off-target side effects.

For bio-pharmaceutical use of micelles as delivery agents, it is desirable to adopt materials regarded as generally safe for drug formulations. In this context, a simple, efficient, and robust method to diversify the structure of micelle-based vectors is of significance in advancing the rational design of effective non-cationic ON vectors, as well as micelle-based peptide and small molecule delivery agents.

### SUMMARY OF THE INVENTION

The invention is directed to a novel form of micelles formed by co-assembly of amphiphilic polymers or copolymers and an optional hydrophobic homopolymer or small molecule and having incorporated therein oligonucleotides, peptides or small molecules. The micelles of the invention address many of the issues associated with the use of micelles to deliver oligonucleotides, peptides and small molecules as therapeutic and diagnostic agents.

In one aspect of the invention, there is provided an oligo-loaded amphiphilic micelle, said micelle being co-assembled from (1) a first amphiphilic copolymer comprising a hydrophobic polymer coupled to an oligonucleotide and (2) a second amphiphilic copolymer, such as PCL coupled to polyethylene glycol (PEG) or PCL coupled to poly(sulfobetaine methacrylate) (PSBMA), and (3) an optional hydrophobic homopolymer or hydrophobic small molecule, wherein the density of hydrophilic polymer on the outer surface of the micelle is in the range of from 1×10¹² to 3×10¹⁴ hydrophilic polymer/cm². In certain embodiments, the micelles are co-assembled from oligonucleotide-b-poly(ε-caprolactone) (oligonuc-b-PCL) and polyethylene glycol-b-PCL (PEG-b-PCL) and PCL homopolymer. In certain embodiments, the oligonucleotide is 2 to 50 bases in length. In certain embodiments, the oligonucleotide is complimentary to and hybridizes to a target polynucleotide. In certain embodiments, the oligonucleotide is DNA or RNA and may be single stranded or double stranded.

In another aspect of the invention, there is provided an amphiphilic peptide-loaded micelle, said micelle being co-assembled from (1) a first amphiphilic polymer comprising a hydrophobic polymer coupled to a peptide and (2) a second amphiphilic polymer, such as PCL coupled to polyethylene glycol (PEG) and (3) an optional hydrophobic homopolymer or hydrophobic small molecule, wherein the density of hydrophilic polymer on the outer surface of the micelle is in the range of from 1×10¹² to 3×10¹⁴ hydrophilic polymer/cm². In certain embodiments, the peptide is 2 to 100 amino acids in length.

In another aspect of the invention, there is provided an amphiphilic micelle, loaded with a small molecule, said micelle being co-assembled from (1) an amphiphilic polymer(s) and (2) a hydrophobic small molecule, wherein the density of hydrophilic polymer on the outer surface of the micelle is in the range of from 1×10¹² to 3×10¹⁴ hydrophilic polymer/cm². In certain embodiments, the hydrophobic small molecule is a therapeutic agent. The small molecule is associated with the micelles through non-covalent interactions, such as van der Waals forces. In other embodiments the small molecule has a molecular weight in the range of from 100 to 1000 Da.

In certain embodiments, the micelles are co-assembled from oligo-b-poly(ε-caprolactone) (oligo-b-PCL) and polyethylene glycol-b-PCL (PEG-b-PCL) and PCL homopolymer. In other embodiments, the micelles are co-assembled from peptide-b-poly(ε-caprolactone) (peptide-b-PCL) and polyethylene glycol-b-PCL (PEG-b-PCL) and PCL homopolymer.

In another aspect, there is provided a composition comprising an amphiphilic micelle, said micelle being co-assembled from a first amphiphilic polymer comprising a hydrophobic polymer coupled to an oligonucleotide or peptide; a second amphiphilic polymer, such as PCL coupled to polyethylene glycol (PEG) or poly(sulfobetaine methacrylate) PSBMA; and an optional homopolymer or small molecule and an optional carrier, such as a therapeutically acceptable carrier.

In another aspect, there are provided methods for inhibiting expression of a gene product encoded by a target polynucleotide, said method comprising contacting a target polynucleotide with a micelle of the invention, wherein the micelle comprises an oligonucleotide that is complimentary to and hybridizes to at least a portion of the target polynucleotide. In various embodiments, expression of the gene product is inhibited in vivo and in other embodiments, expression of the gene product is inhibited in vitro.

In yet another aspect, methods are provided for promoting the cellular uptake of an oligonucleotide, peptide or small molecule in a subject or biological sample, said methods comprising delivering an oligonucleotide-, peptide- or small molecule-loaded micelle, respectively, of the invention to the subject or the biological sample in an effective amount for cellular uptake of the oligonucleotide, peptide or small molecule. An effective amount of loaded micelles in this context is from 1 pM/kg micelle to 10 μM/kg micelle.

Another aspect of the invention provides a method of detecting the presence of a target polynucleotide in a subject or in a tissue sample obtained from a subject, said method comprising contacting the target polynucleotide with an oligonucleotide-containing micelle of the invention, wherein the oligonucleotide is complementary to and hybridizes to at least a portion of the target polynucleotide.

Another aspect of the invention provides a method of delivering an oligonucleotide, peptide or small molecule to a target site in vivo, the method comprising administering a composition comprising an oligo-loaded or a peptide-loaded or small molecule-loaded micelle to a subject. In this aspect, the oligo-loaded micelle comprises a hydrophobic polymer coupled to an oligonucleotide, the peptide-loaded micelle comprises a hydrophobic polymer coupled to the peptide; and each loaded micelle comprises a second amphiphilic polymer, such as a PCL block copolymer coupled to polyethylene glycol (PEG); and a PCL homopolymer and an optional carrier, such as a therapeutically acceptable carrier. In this aspect, a small molecule-loaded micelle comprises an amphiphilic polymer associated with the small molecule through non-covalent forces, such as van der Waals forces; an optional hydrophobic polymer; and an optional carrier, such as a therapeutically acceptable carrier.

Consistent with the various aspects of the invention described herein, oligonucleotide-loaded micelles of the invention may comprise a plurality of identical oligonucleotides or a plurality of identical oligonucleotides and at least one distinct (different) oligonucleotide coupled to PCL or other hydrophobic polymer. In other embodiments of the various aspects of the invention described herein, oligonucleotide-loaded micelles are provided in which the micelles have at least two different oligonucleotides coupled thereto, as described herein below. In certain embodiments, the different oligonucleotides hybridize to different regions on the same target polynucleotide or hybridize to different target polynucleotides. In certain embodiments of the various aspects of the invention, the target polynucleotide is a bacterial polynucleotide, viral polynucleotide, fungal polynucleotide or disease- or disorder-specific polynucleotide, such as a polynucleotide associated with cancer, for example. The target polynucleotide may be DNA or RNA, single stranded or double stranded.

Also consistent with the various aspects of the invention described herein, peptide- or small molecule-loaded micelles of the invention may comprise a plurality of identical peptides or small molecules, respectively or a plurality of identical peptides or small molecules and at least one distinct (different) peptide or small molecule, respectively, coupled to PCL or other hydrophobic polymer. In other embodiments of the various aspects of the invention described herein, peptide- or small molecule-loaded micelles are provided in which the amphiphilic micelles have at least two different peptides or small molecules, respectively, associated with an amphiphilic polymer or copolymer, as described herein below. In certain embodiments, the peptide or small molecules is a therapeutic agent.

### BRIEF DESCRIPTION OF THE DRAWINGS

FIG. 1 is an ¹H NMR spectrum of PCL-N₃ in CDCl₃.

FIG. 2 is an ¹³C NMR spectrum of PCL-N₃ in CDCl₃.

FIG. 3 is a FTIR spectrum of PCL-N3. The arrow indicates azide stretching vibration.

FIG. 4 shows an agarose gel (1%) electrophoresis of free DNA and various micelle compositions. Certain samples with high 10 kDa PEG content migrate in the opposite direction of free DNA due to the transient interactions between PEG and passing cations.

FIG. 5 is a reverse-phase HPLC chromatogram of the reaction mixture for DNA/PCL coupling reaction. The conjugate has baseline separation from unreacted DNA.

FIGS. 6A and B show an agarose gel (1%) electrophoresis of DNA-b-PCL micelles and free DNA. (A) Ethidium bromide-stained unlabeled DNA. (B) Cy3-labeled DNA.

FIG. 7(A)-(E) show the schematics and results of a fluoresecence assay used for quantifying DNA hybridization. (A) Schematics of the fluorescence assay; (B, D) hybridization kinetics for free DNA versus two- and three-component nanoparticles; (C, E) Relationship between PEG density and the percentage of fast-hybridizing DNA population.

FIG. 8A-E shows the schematics and results of a study of enzyme accessibility within the micelle. (A) Schematics of the fluorescence assay used for monitoring the kinetics of DNA degradation by DNase1; (B, D) DNA degradation kinetics for free DNA versus two- and three-component nanoparticles; (C, E) Relationship between PEG density and the DNA nuclease half-life.

FIG. 9A-C show the correlation of micelle structure and antisense gene silencing efficacy. (A, B) Flow cytometry measurements of SKOV3 cells treated with Cy₃-labeled free DNA and micellar nanoparticles (conc. 1 μM DNA); (C) corresponding confocal fluorescence images. Scale bar: 20 μm.

FIGS. 10A and B: (A) is a bar graph showing efficacy for antisense gene knockdown using micelles and controls (1 μM DNA). (B) is a bar graph showing dose response of two co-assembled micelles with high efficacy.

FIGS. 11A and B: A and B are graphs showing the results of a MTT cytotoxicity assay for SKOV3 cells treated with free DNA, micellar nanoparticles (A) or Lipofectin 2K-complexed DNA (B).

### DETAILED DESCRIPTION OF THE INVENTION

The present invention addresses many of the issues associated with the use of micelles to deliver oligonucleotides, peptides and small molecules as therapeutic agents and/or diagnostics. Micelles are structures composed of a monolayer of amphipathic molecules, which tend to arrange themselves in such a manner that the inner core of these structures is hydrophobic and the outer layers are hydrophilic in nature. It is demonstrated herein that stability and cellular uptake of oligonucleotide-, peptide- or small molecule-loaded micelles which are co-assembled from amphiphilic polymers and optional hydrophobic homopolymer or small molecule are affected by adjusting the density and length of the micelle surface moieties. Oligonucleotide hybridization availability of oligo-loaded micelles also can be adjusted by adjusting the density and length of the micelle surface moieties. The density of the surface moieties (spacing between the surface moieties) may be adjusted by the addition of an optional hydrophobic polymer or hydrophobic small molecule to the amphiphilic polymer composition.

The inventors have developed a novel form of amphiphilic polymer-based nanoparticle micelles, e.g., PEG-b-polymer-based nanoparticle micelles that are loaded with oligonucleotides, peptides or small molecules, which have tunable polymer length (molecular weight) and density at the micelle surface. The nanoparticle micelles comprise two amphiphilic polymers, which provide a hydrophobic core that is useful for encapsulation of hydrophobic molecules, and a hydrophilic corona that consists of oligonucleotides or peptides, and a shielding polymer such as polyethylene glycol (PEG). By adjusting the relative length and/or adjusting the density of the surface moieties of the micelles through, for example, the addition of a diluting hydrophobic homopolymer or hydrophobic small molecule to the micellar core, the resulting micelles provide protection of the oligonucleotide in the corona from enzymatic degradation (e.g., nuclease stability) and significant cellular uptake without affecting the ability of the oligonucleotide to hybridize to the target polynucleotide.

The inventors have found that, when an intermediate density of the amphiphilic micelle is achieved, nuclease access of an oligonucleotide is greatly hindered, while formation of double-stranded DNA is nearly unaffected. Micelles showing the highest selectivity (good nuclease resistance while still maintaining hybridization kinetics) also exhibit the highest antisense gene silencing efficacies.

Any biocompatible, non-biofouling amphiphilic polymer that does not interact with protein (e.g., exhibits stealth properties which enable it to avoid recognition by liver receptors and other proteins) can be used to generate the micelles of the invention. (See Laschewsky, A., Polymers, 2014, 6, 1544-1601, incorporated herein). For example, polyethylene glycol (PEG) block copolymers (PEG-b-polymer) may be used, as well as zwitterion polymer-based copolymers such as poly(methacryloyl-L-lysine)-b-polymer, poly(sulfobetaine methacrylate)-b-polymer and poly(carboxybetaine methacrylate)-b-polymer.

The polymer components of the micelles include two amphiphilic polymers and an optional hydrophobic homopolymer. The amphiphilic polymer(s) may be an amphiphilic di-block copolymer, a tri-block copolymer, or other architecture, such as gradient copolymers (as opposed to block copolymer) or combination thereof. Any hydrophobic amphiphilic polymer, and in particular, biocompatible and preferably, biocompatible and biodegradable polymers, may be used to generate the amphiphilic micelles of the invention. For example, the polymers that comprise the oligonucleotide-coupled-polymer or peptide-coupled polymer, and the second amphiphilic -polymer independently each may be poly(lactide-co-glycolide) (PLGA), polylactide (PLA), polyglycolide (PGA), D-lactide, D,L-lactide, L-lactide, D,L-lactide-co-ε-caprolactone, D,L-lactide-co-glycolide-co-ε-caprolactone or a combination thereof. For example, the amphiphilic polymer component of the micelle may be a mixture of a PEG block copolymer (PEG-b-polymer) or another polymer having PEG-like properties, e.g., poly(carboxybetaine methacrylate) or a mixture of PEG-b-polymer and oliogonucleotide-b-polymer (oligo-b-polymer) or peptide-b-polymer (peptide-b-polymer), for example. In each case, the polymers may be the same or different. In all cases the polymers are non-cationic, amphiphilic and preferably, biocompatible and more preferably, biodegradable.

The properties of the micelles are adjusted, in part, by the selection of the length (or molecular weight) of the hydrophilic segment of the amphiphilic polymer used to assemble the micelles. The hydrophilic segment may range in size from about 0.1 to about 1,000 kDa, preferably from about 2-100 kDa, more preferably from 5-80 kDa and more preferably from about 5-50 kDa. The micelles may be formed from a mixture of amphiphilic polymers of varying lengths within this size range or may be assembled from amphiphilic polymers of a single length within this size range.

The density of the amphiphilic polymers on the surface of the micelles also may be adjusted for different payloads, i.e., oligonucleotide, peptide or small molecules. The density of micelle surface moieties is generally in the range of from 1.0×10¹² to 3.0×10¹⁴ hydrophilic polymer/cm², e.g., 1.0×10¹² to 3.0×10¹⁴ PEG/cm², such as for example, 4.5×10¹³-6.3×10¹³ PEG/cm². The density refers to that at the core-shell interface of the micelle.

The density of (spacing between) the surface moieties of the micelles may be adjusted by addition of a hydrophobic homopolymer or hydrophobic small molecule to the micelle core, which decreases the surface density. Any homopolymer, preferably a biocompatible, and most preferably a biocompatible and biodegradable polymer can be used to adjust the density of the micelle surface moieties. For example, polycaprolactone (PCL) may be added to the micelle composition to adjust the density of surface moieties. The homopolymer may be the same as the amphiphilic polymer coupled to oligonucleotide or peptide (if the payload is oligonucleotide or peptide, respectively). In some embodiments, the homopolymer may be a different biocompatible and/or biodegradable polymer than used to form the micelles or it may be a hydrophobic small molecule, such as a drug for example. Non-limiting examples of hydrophobic small molecules that may be included in the micelles to adjust surface density include vitamin E, oils, cholesterol, paclitaxel, camptothecin, doxorubicin and the like. In the case of small molecule drugs, the “homopolymer” has a dual function as a drug and density adjusting molecule. For example, when the small hydrophobic molecule is cholesterol or the drug camptothecin is used to adjust surface density of an oligonucleotide-loaded micelle, the composition of the micelle is amphiphilic polymers with oligonucleotide and PEG as the hydrophilic segments, and cholesterol or camptothecin.

The optional hydrophobic homopolymer or hydrophobic small molecule may be included in the micelles in an amount of from 0% to about 95 weight percent of the micelle, more preferably from about 1 to 80%. In general, the molecular weight of the homopolymer or small molecules used to adjust the density of surface moieties is in the range of from 100 Da to about 1,000 kDa. For example, the molecular weight of small molecules is generally in the range of from 100 to 1000 Da, and the molecular weight of homopolymer is generally in the range of from about 1000 Da to 1000 KDa.

Co-assembly of the amphiphile and the homopolymer or small molecule may be achieved via nanoprecipitation (e.g., gradual solvent exchange from DMSO to Nanopure™ water), for example. The homopolymer or small molecule contributes to the micellar core volume, but not the shell, thus modulating the density of micelle surface moieties.

In those embodiments of the various aspects of the invention where the micelles are loaded with an oligonucleotide, the oligonucleotide component of the micelle can be single stranded or double stranded DNA or double stranded RNA. Oligonucleotides contemplated for use in the invention include those which modulate expression of a gene product expressed by a target polynucleotide. Accordingly, antisense oligonucleotides which hybridize to a target polynucleotide and inhibit translation, siRNA oligonucleotides which hybridize to a target polynucleotide and initiate an RNAse activity (for example RNAse H), triple helix forming oligonucleotides which hybridize to double-stranded polynucleotides and inhibit transcription, and ribozymes which hybridize to a target polynucleotide and inhibit translation, are contemplated.

Each oligonucleotide-loaded micelle utilized in the compositions and methods provided herein has a plurality of oligonucleotides covalently attached to the polymer core. As a result, micelles have the ability to bind to (hybridize with) a plurality of target polynucleotides having a sufficiently complementary sequence. For example, if a specific mRNA is targeted, an oligonucleotide-loaded micelle has the ability to hybridize with multiple copies of the same transcript. In one embodiment, methods are provided wherein the oligo-loaded micelle is functionalized with identical oligonucleotides, i.e., each oligonucleotide has the same length and the same sequence. In other embodiments, the micelle is functionalized with two or more oligonucleotides, which are not identical, i.e., at least one of the attached oligonucleotides differs from at least one other attached oligonucleotide in that it has a different length and/or a different sequence or modification. In embodiments where different oligonucleotides are attached to the polymers forming the micelle core, these different oligonucleotides hybridize with the same single target polynucleotide, but at different locations, or hybridize with different target polynucleotides which encode different gene products. Accordingly, in various aspects of the invention, a single oligo-loaded micelle may be used to inhibit expression of more than one gene product. Oligonucleotides are thus used to target specific polynucleotides, whether at one or more specific regions in the target polynucleotide, or over the entire length of the target polynucleotide as the need may be to affect a desired level of inhibition of gene expression.

In those embodiments of the invention where the micelles are loaded with oligonucleotides, the range of oligonucleotide-coupled polymer to amphiphilic polymer, e.g., PEG-b-polymer contained in the micelle is in the range of from 0.00001:1 to 1:0.00001, more preferably in the range of from 2:1 to 1:50. In such embodiments, the amount of oligonucleotide is generally in the range of from about 1 pM to about 100 μM/kg micelle. This ratio may be adjusted depending on the ultimate application of the oligo-loaded micelles, e.g., as diagnostics or as therapeutics. In general, less oligonucleotide is necessary for diagnostic applications than for therapeutic applications. For example, diagnostic applications may include from 1 pM/kg to 1 nM/kg, while therapeutics may include a higher concentration of oligonucleotides, e.g., from 1 nM/kg to100 μM.

“Hybridize” and “hybridization” are used herein to mean an interaction between two strands of nucleic acids by hydrogen bonds in accordance with the rules of Watson-Crick DNA complementarity, Hoogstein binding, or other sequence-specific binding known in the art. Hybridization can be performed under different stringency conditions known in the art. Under appropriate stringency conditions, hybridization between the two complementary strands could reach about 60% or above, about 70% or above, about 80% or above, about 90% or above, about 95% or above, about 96% or above, about 97% or above, about 98% or above, or about 99% or above in the reactions. It will be understood by those of skill in the art that the degree of hybridization is less significant in the disclosed technology than a resulting degree of inhibition of gene product expression.

The oligonucleotides are designed with knowledge of the target sequence or sequences. Methods of making oligonucleotides of a predetermined sequence are well-known. See, for example, Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are contemplated for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically.

Alternatively, oligonucleotides are selected from a library. Preparation of libraries of this type is well known in the art. See, for example, Oligonucleotide libraries: United States Patent Application 20050214782, published Sep. 29, 2005.

In another aspect, methods and compositions are provided wherein the oligonucleotide is bound to polymer of the micelle in such a way that the oligonucleotide is released from the micelle after the micelle enters a cell. In general, an oligonucleotide can be released from the micelle using either chemical methods, photon release (i.e., irradiating cells in which uptake of micelles has occurred using electromagnetic wavelength selected on the basis of micelle size, changes in ionic or acid/base environment or a cleavable bond or use of an acid-labile or redox-labile linker, for example.

In one embodiment of this aspect, the oligonucleotide is attached to a polymer via a redox-labile moiety and once the micelle is taken into the cell, the oligonucleotide is released from the polymer. For example, an RNA-loaded micelle can be synthesized to feature a redox-labile linker. Once the micelle enters the cells, the disulfide bond may be cleaved by glutathione inside the cells and free dsRNA is released to perform gene regulation functionality. This aspect is particularly useful in instances where the intent is to saturate the cell with for example, an siRNA and release from the micelle would improve kinetics and resolve potential steric hindrance problems. Preparation and use of RNAi for modulating gene expression are well known in the art.

In general, the oligonucleotide, or modified form thereof, is from about 2 to about 60 nucleotides in length. It is also contemplated that the oligonucleotide is about 2 to about 50 nucleotides in length, about 2 to about 45 nucleotides in length, about 2 to about 40 nucleotides in length, about 2 to about 40 nucleotides in length, about 2 to about 35 nucleotides in length about 2 to about 30 nucleotides in length, about 2 to about 25 nucleotides in length, about 2 to about 20 nucleotides in length, about 2 to about 15 nucleotides in length, about 2 to about 10 nucleotides in length, and all oligonucleotides intermediate in length of the sizes specifically disclosed to the extent that the oligonucleotide is able to achieve the desired result. Accordingly, oligonucleotides of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length are contemplated.

In various embodiments of this aspect of the invention, the micelles, compositions and their use include use of an oligonucleotide which is 100% complementary to the target polynucleotide, i.e., a perfect match or 100% sequence identity over the entire length of the oligonucleotide, while in other aspects, the oligonucleotide is at least about 95% complementary to the polynucleotide over the length of the oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to the polynucleotide over the length of the oligonucleotide to the extent that the oligonucleotide is able to achieve the desired degree of inhibition of a target gene product.

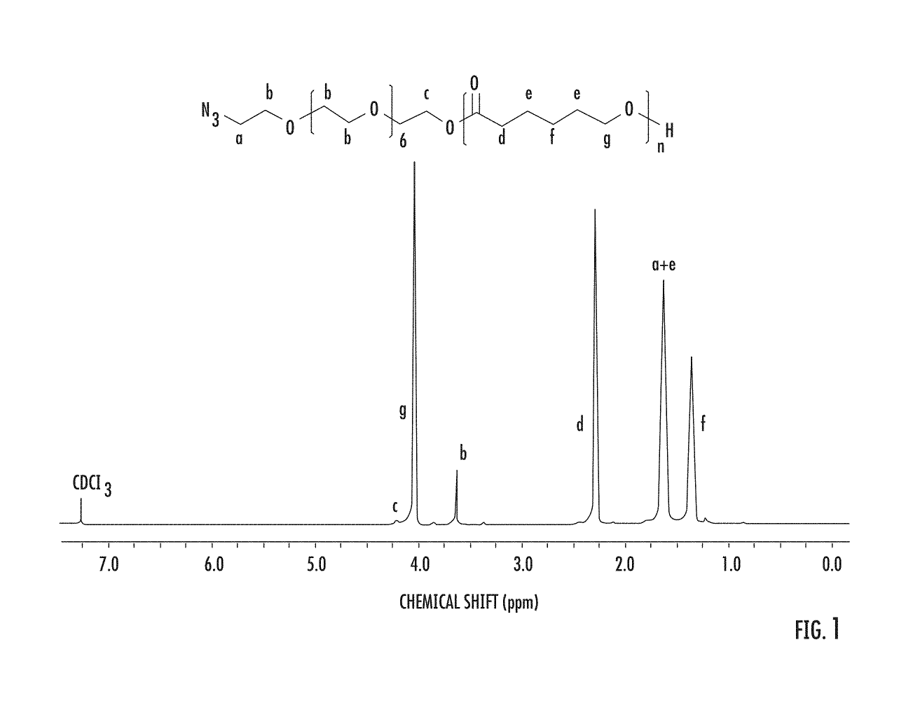

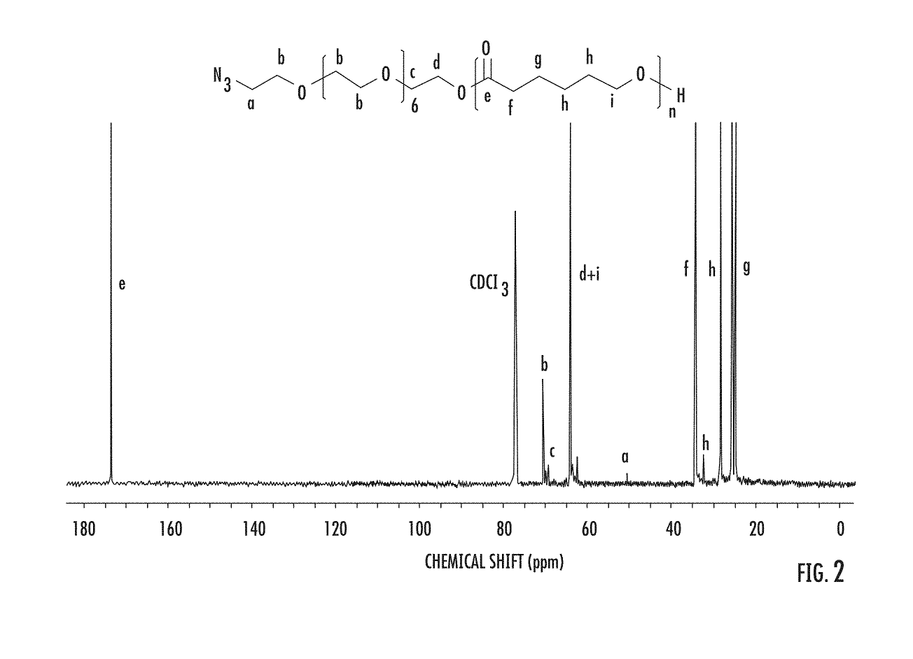

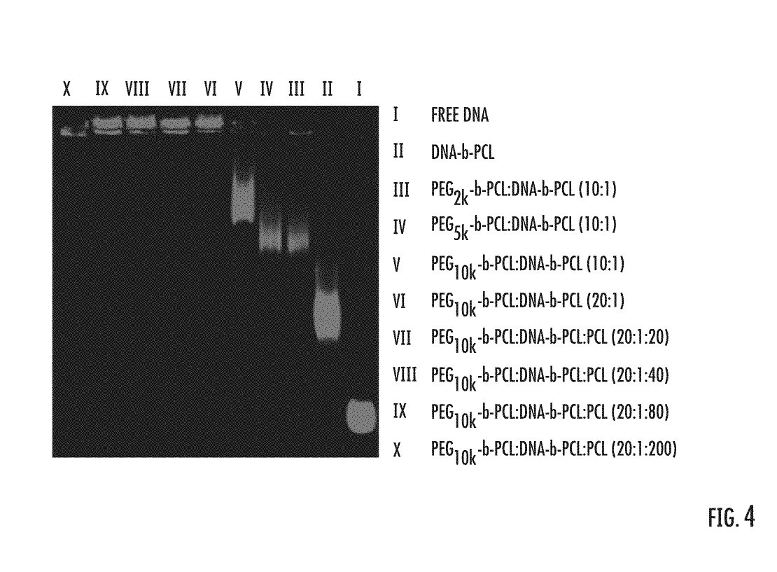

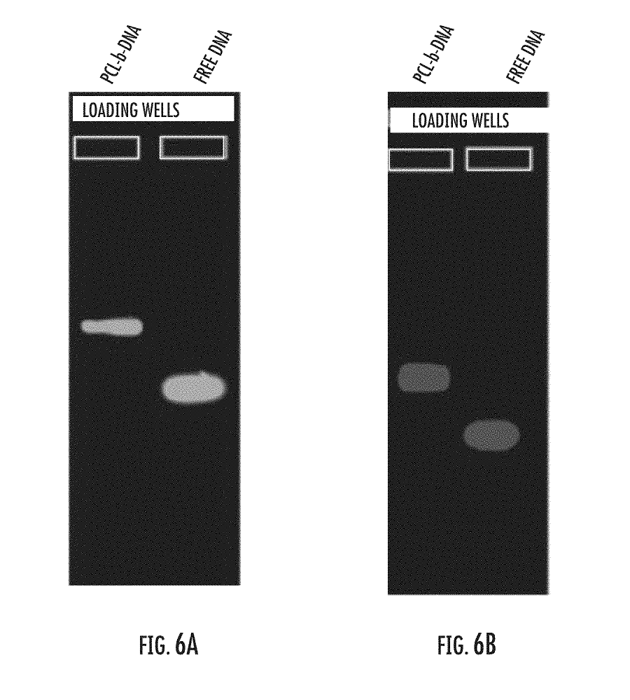

Oligonucleotides contemplated for use in the methods include those bound to the polymer core through any means. The nucleic acid-b-polymer amphiphile is synthesized by any suitable method for the particular type of polymer being used. For example, for an oligonucleotide-b-PCL amphiphile, synthesis may be a two-step process. First, azide-terminated PCL may be prepared via ring-opening polymerization (ROP) of ε-caprilactone (CL) using O-(2-azidoethyl)heptaethylene glycol as the initiator (for 1H/13C NMR and infrared spectra, see FIGS. 1-3). Subsequently, an oligonucleotide (ON) with a 5′ dibenzocyclooctyl (DBCO) group and a 3′ Cy3 reporter (See Table 1) is coupled to the azide-capped PCL through copper-free click chemistry in dimethyl sulfoxide (DMSO): water mixture (9:1 v:v). Unreacted PCL and DNA are removed by dialysis and reverse-phase HPLC, respectively, to yield pure conjugates (˜85% conjugation yield) as determined by agarose gel electrophoresis (FIGS. 4, 5 and 6).

Regardless of the means by which the oligonucleotide is attached to the polymer, attachment in various aspects is effected through a 5′ linkage, a 3′ linkage, some type of internal linkage, or any combination of these attachments. For example, oligonucleotides or a subset of oligonucleotides in any particular micelle can be covalently attached at their 5′ end to a polymer via an amine. In this case, the oligonucleotide is designed to have a 5′ amine group. For example, the oligo-b-polymer structure is synthesized by coupling amine-modified DNA to a block polymer containing N-hydroxyl succinimide (NHS) groups in an aqueous bicarbonate buffer. Alternatively, cyclooctyne-mediated copper-free click chemistry can be used in place of the amidation reaction, resulting in near-quantitative yields. To achieve the coupling, the oligonucleotide strand can be modified with 5′ dibenzocyclooctyne (DBCO) group, while the polymer bears the azide groups.

Similarly, the oligonucleotides or a subset of oligonucleotides in any particular micelle can be linked to the polymer at the 3′ end of the oligonucleotide.

Alternatively, all or some of the oligonucleotides in a micelle can be attached to the polymer via an internal thymidine nucleobase of the oligonucleotide. In this case, solid phase DNA synthesis can be used to incorporate amine or alkyne modification at the 5 position of thymine (T) bases, which can be used to couple to polymers from any location of the DNA. The modification site of the T base point into the major groove, and does not interfere with the base from participating in hybridization.

The oligonucleotides can be attached to polymer at any position, such as at either end of the polymer backbone or along the internal portion of the polymer backbone. Polymers with different DNA attachment points, DNA strands with varying polymer attachment points, and polymers having multiple, randomly embedded attachment points throughout the polymer are contemplated. With current polymer synthetic methodology (ring opening metathesis polymerization), multi-block polymers are readily synthesized by sequential addition of macromonomers. Each step can be monitored to ensure that the monomer is fully consumed before addition of the next monomer. It is understood that different oligonucleotides can be attached at different locations along the length of the polymer backbone. Preferably, but not necessarily, the oligonucleotides are attached at or near either end of the polymer.

In certain embodiments, a detectable label may incorporated at either end of the oligonucleotide to facilitate tracking and quantification of the oligo-loaded micelles.

Coupling of the oligonucleotides to the hydrophobic polymer via a 5′ linkage, 3′ linkage or via internal nucleobases as disclosed herein provides micelles containing a plurality of oligonucleotides. The amount of coupled oligonucleotides can be adjusted by manipulating the linking chemistry. For example, amphiphilic polymers containing 2, 3, 4, 5, 6, 7 or more oligonucleotides, which may be the same or different, can be achieved.

In various aspects of the invention, the target polynucleotide is either eukaryotic, prokaryotic, or viral. In various embodiments, the target polynucleotide is an mRNA encoding a gene product and translation of the gene product is inhibited by the oligo-loaded micelle, or the target is a gene encoding a gene product and transcription of the gene product is inhibited. The target polynucleotide may be a DNA that encodes a gene product being inhibited or may be complementary to a coding region for a gene product. In still other embodiments, the target DNA encodes a regulatory element necessary for expression of a gene product. “Regulatory elements” include, but are not limited to enhancers, promoters, silencers, polyadenylation signals, regulatory protein binding elements, regulatory introns, ribosome entry sites, and the like. In still, other embodiments the target polynucleotide is a sequence which is required for endogenous replication.

DNA target regions include any portion of the target nucleic acid, such as the 5′ untranslated region (5′UTR) of a gene, the portion of an mRNA in the 5′ direction from the translation initiation codon, including nucleotides between the 5′ cap site and the translation initiation codon of an mRNA (or corresponding nucleotides on the gene), and the 3′ untranslated region (3′UTR), the portion of an mRNA in the 3′ direction from the translation termination codon, including nucleotides between the translation termination codon and 3′ end of an mRNA (or corresponding nucleotides on the gene).

In some embodiments of the various aspects of the invention, the target nucleic acid is a gene or RNA transcript specific to a cancer cell or which is over-expressed in cancer cells, such as HER2, for example, which is over-expressed in several types of cancer, such as breast cancer, ovarian and stomach cancer.

For prokaryotic target polynucleotides, the polynucleotide is genomic DNA or RNA. For eukaryotic target polynucleotides, the polynucleotide is an animal polynucleotide, a plant polynucleotide or fungal polynucleotide, including yeast polynucleotides. The target polynucleotide is either a genomic DNA or RNA. In certain embodiments, the target polynucleotide is a mitochondrial polynucleotide. For viral target polynucleotides, the polynucleotide is viral genomic RNA or transcribed RNA or viral genomic DNA.

Accordingly, the oligo-loaded micelles described herein may be used to diagnose, prevent, treat or manage certain diseases or bodily conditions. In some cases, the oligo-loaded micelles are both a therapeutic agent and a diagnostic agent. Therapeutic methods provided herein embrace those which result in essentially any degree of inhibition of expression of a target gene product.

In some embodiments, oligonucleotide-loaded micelles described herein may be used as intracellular diagnostic agents. The ability to deliver nucleic acids intact to the cell cytoplasm provides an opportunity to not only regulate RNA targets, but also to detect them. For instance, in some embodiments, delivery of an oligo-loaded micelle having 3′ and/or 5′ detectable markers is used to detect the presence of target RNA. In other embodiments, the micelle may be designed with oligonucleotides to detect the presence of intracellular proteins (e.g., aptamers) or small molecules through changes in fluorescence that occur due to target protein or small molecule binding, respectively. The micelles described herein may be made to deliver nucleic acid sensors for a broad range of biomolecules that provide a convenient readout of their presence, for example, through increased fluorescence upon target molecule binding.

For the embodiments of the various aspects of the invention where peptide-loaded micelles are generated and/or used as therapeutic agents or diagnostic agent, the peptide micelles are generated in the same general fashion as described above for oligo-loaded micelles. The composition of the peptide micelles would be amphiphilic polymer, peptide-coupled amphiphilic polymer, and optional hydrophobic homopolymer (e.g., PCL) or small molecule. The preparation procedure is similar to that of the oligonucleotide micelles as described above. For example, micelle components, e.g., PEG-b-PCL, peptide-b-PCL and PCL/cholesterol/camptothecin, are weighed to give a desired molar ratio, and are fully dissolved in DMSO under mixing. Then, water is added to the mixture using a syringe pump, for example, over a period of time (e.g. 2 h). Subsequently, the mixture is dialysed against water to remove the organic residues. In general, the peptides incorporated into the micelles of the invention are in the range of from 2 to 100 amino acids in length, preferably from 2 to 75 amino acids, and more preferably from 3 to 50 amino acids in length. The peptides may be modified. Such peptide modifications include for example, attachment of fluorophores and/or quenchers, such as emitting and quenching dyes for FRET and other dyes; attachment of linkers, spacers, or PEG, for example; inclusion of unnatural amino acids, D-stereoisomers; post-translational modifications such as myristoylation, palmitoylation, glycosylation, phosphorylation, citrullination, methylation, succinylation, and the like; isotopic labelling (heavy) of amino acids with, for example, Carbon 13 (¹³C), Nitrogen 15 (¹⁵N) or Deuterium (²H); conjugation to immunogens, carrier proteins, lipids, nucleic acids, and the like; dimerization; cyclization; peptide stapling; biotinylation; carboxymethylation (CAM); addition of protecting groups; bioconjugation; and the like.

The range of peptide-coupled polymer to amphiphilic polymer, e.g., PEG-b-polymer contained in the micelle is in the range of from 0.00001:1 to 1:0.00001, more preferably in the range of from 2:1 to 1:50. In such embodiments, the amount of peptide is generally in the range of from about 1 pM to about 100 μM/kg micelle. This ratio may be adjusted depending on the ultimate application of the peptide-loaded micelles, e.g., as diagnostics or as therapeutics. In general, less peptide is necessary for diagnostic applications than for therapeutic applications. For example, diagnostic applications may include peptide from 1 pM/kg to 1 nM/kg, while therapeutics may include a higher concentration of peptide, e.g., from 1 nM/kg to100 μM.

Where it is necessary to release the molecules loaded into or on the surface of the micelles, e.g., peptides, siRNA, or small molecules, a release mechanism may be used, as described herein above. For example, the peptides may have release mechanisms that are similar to those used with RNA to release the peptide after delivery to the target site. For example, for release of a peptide so that it might engage with a protein complex target, a disulfide bond between the polymer (e.g., PCL) and the peptide may be introduced. The disulfide bond will be cleaved by the reductive intracellular environment such as the intracellular environment of tumor cells, which contain a high concentration of glutathione, resulting in the release of the peptide.

In some embodiments of the invention, peptide- or small molecule-loaded micelles described herein may be used as diagnostic agents. The advantage of the of the invention is that the intermediate density on the micelle surface shields the oligonucleotide or oligopeptide from unwanted, non-specific interactions, and the intended specific interaction is unhindered. Thus, the micelles herein are more specific and can potentially lead to more accurate diagnostic results.

The oligo-, peptide- and small molecule-loaded micelles of the invention may be used in “pharmaceutical compositions” or “pharmaceutically acceptable” compositions, which comprise a therapeutically effective amount of one or more of the micelles described herein, formulated together with one or more pharmaceutically acceptable carriers, additives, and/or diluents. The pharmaceutical compositions described herein may be useful for diagnosing, preventing, treating or managing a disease or bodily conditions such as cancer or bacterial or viral infection, for example. It should be understood that any micelle described herein can be used in such pharmaceutical compositions.

Pharmaceutical compositions containing the micelles described herein may be specially formulated for administration in solid or liquid form, including those adapted for oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

As used herein, the term “pharmaceutically acceptable” refers to those structures, materials, compositions, and/or dosage forms which are, within the scope of sound medical judgment, suitable for use in contact with the tissues of human beings and animals without excessive toxicity, irritation, allergic response, or other problems or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the micelle from one organ, or portion of the body, to another organ, or portion of the body. Each carrier is “acceptable” in the sense of being compatible with the other ingredients of the formulation and not injurious to the patient when administered in doses sufficient to provide a therapeutically effective amount of the loaded micelle. Non-limiting examples of materials that can serve as pharmaceutically-acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The loaded micelles and compositions containing loaded micelles may be orally administered, parenterally administered, subcutaneously administered, and/or intravenously administered for carrying out the methods of the invention. In certain embodiments, a loaded micelle or pharmaceutical composition containing a loaded micelle s administered orally. In other embodiments, the loaded micelle or pharmaceutical composition containing a loaded micelle is administered intravenously or via injection into a target site such as a tumor or muscle. Alternative routes of administration include sublingual, intramuscular, and transdermal administrations.

In some embodiments of the invention, the loaded micelle or composition containing a loaded micelle is applied to a biological sample, such as a blood sample or other tissue obtained from a subject, such as a human or other mammal. In this case, the oligo, peptide or small molecule which is loaded into the micelle is optionally labelled with one or more detectable labels.

Also provided are kits for inhibiting gene expression of a target polynucleotide. In one embodiment of this aspect, the kit contains at least one type of oligo-loaded micelle as described herein or a plurality of types of oligo-loaded micelles providing a plurality of different oligonucleotides as described herein attached to a polymeric micelle. The oligonucleotides on the first type of oligo-loaded micelles have one or more sequences complementary (or sufficiently complementary as disclosed herein) to one or more sequences of a first portion of a target polynucleotide. The kit optionally includes one or more additional type of oligo-loaded micelles which have a sequence complementary to one or more sequences of a second portion of the target polynucleotide or to a second target sequence.

In some embodiments of the kits provided, oligonucleotides include a detectable label or the kit includes a detectable label which can be attached to the oligonucleotides or the amphiphilic micelle.

### Definitions

The terms “oligonucleotide” and “oligo” are used interchangeably herein and are used herein to include modified forms as discussed herein below, as well as those known in the art to regulate gene expression.

The terms “oligo-loaded micelle” or “peptide-loaded micelle” are used herein to mean a polymeric micelle co-assembled from two amphiphilic polymers and an optional hydrophobic homopolymer or optional hydrophobic small molecule, wherein one of the amphiphilic polymers comprises a nucleic acid, oligonucleotide or peptide, respectively, as the hydrophilic segment, and the other comprises a protein binding-resistant polymer, e.g. polyethylene glycol as the hydrophilic segment. Such Micelles are generally “surface-loaded” micelles, where nucleic acid, oligonucleotide or peptide is generally present on the surface of the micelle.

The term “small molecule loaded micelle” is used to herein to mean a polymeric micelle co-assembled from two amphiphilic polymers and an optional hydrophobic homopolymer, wherein a hydrophobic small molecule, such as a drug, is incorporated into the core of the micelle via non-covalent interactions with the polymers of the micelle core. Small molecule-loaded micelles are “core-loaded” micelles.

As used herein, a “small molecule” includes low molecular weight (<1000 daltons) organic compounds with a size on the order of ˜1 nm and which are hydrophobic.

As used herein, the term amphiphilic polymer or amphiphilic compound means a polymer or organic compound combining both hydrophilic and hydrophobic properties.

The term “coupled” is used herein to mean covalent binding, for example.

### EXAMPLES

### Example 1

### Materials and Methods

1. Materials

ε-Caprolactone (ε-CL, 99%, Acros Organics Co.) was dried over calcium hydride for 24 h and purified by distillation under reduced pressure prior to use. Monomethoxy poly(ethylene glycol) (PEG, Mn=2, 5, 10 kDa, PDI<1.1) was purchased from Sigma-Aldrich Co., and dried by azeotropic distillation in the presence of dry toluene. Phorsphoramidites and supplies for DNA synthesis were purchased from Glen Research Co. Human SKOV3 cancer cell line was purchased from American Type Culture Collection (Rockville, Md., USA). All other reagents and solvents were purchased from Sigma-Aldrich Co., VWR International LLC., or Fisher Scientific Inc., and used without further purification unless otherwise mentioned.

2. Measurements

All NMR spectra were recorded on a Varian 400 MHz NMR spectrometer (Varian Inc., CA, USA) with deuterated chloroform (CDCl3) as the solvent. Fourier transform infrared (FTIR) spectra were recorded on a Bruker Tensor FT-IR spectrometer (Bruker Corporation, MA, USA) using a KBr plate as sample holder. N, N-Dimethylformamide (DMF) gel permeation chromatograph was performed on a TOSOH EcoSEC HLC-8320 GPC system (Tokyo, Japan) equipped with a TSKGel GMHHR-H, 7.8×300 mm column and RI/UV-Vis detectors. HPLC-grade DMF with 0.04 M LiBr was used as the mobile phase at a flow rate of 0.5 mL/min. GPC calibration was based on polystyrene standards (706 kDa, 96.4 kDa, 5970 Da, 500 Da). Aqueous GPC measurements were carried out on a Waters Breeze 2 GPC system equipped with an UltrahydrogelTM 500, 7.8×300 mm column and a 2998 PDA detector (Waters Co., MA, USA). Sodium nitrate solution (0.1 M) was used as the eluent running at a flow rate of 0.8 mL/min. MALDI-ToF MS measurements were carried out on a Bruker Microflex LT mass spectrometer (Bruker Daltonics Inc., MA, USA). Reverse phase HPLC was performed using a Waters Breeze 2 HPLC system coupled to a Symmetry C18 3.5 μm, 4.6 Å˜75 mm reverse phase column and a 2998 PDA detector, using TEAA buffer (0.1 M) and HPLC-grade acetonitrile as mobile phases. Gel electrophoresis was performed using 0.5% agarose gel in 0.5× tris/borate/EDTA (TBE) buffer with a running voltage of 100 V. Gel images were acquired on an Alpha Innotech Fluorochem Q imager. Dynamic light scattering (DLS) and zeta potential data were acquired on a Malvern

Zetasizer Nano-ZSP (Malvern, UK). The reported data were the mean of three separate measurements. Transmission electron microscopy (TEM) was performed on a JEOL JEM 1010 electron microscope at a voltage of 80 kV. UV-Vis data were obtained on a Cary 4000 UV-Vis spectrophotometer (Varian Inc., CA, USA). Fluorescence spectroscopy was carried out on a Cary Eclipse fluorescence spectrophotometer (Varian Inc., CA, USA).

3. Methods

3.1 Synthesis of Azide-Terminated Poly(ε-Caprolactone) (PCL-N3)

PCL-N3 was prepared by ring-opening polymerization (ROP) of ε-CL using the O-(2-azidoethyl) heptaethylene glycol as an initiator and Sn(Oct)2 as the catalyst. In a typical polymerization reaction, O-(2-azidoethyl)heptaethylene glycol (0.125 g, 0.316 mmol) and ε-CL (3.16 g, 27.7 mmol) were added to a dried flask and placed under nitrogen atmosphere. Sn(Oct)2 (11.2 mg, 0.0277 mmol) was then added to the reaction mixture via syringe, followed by immediate immersion of the reaction flask into an oil bath at 120° C. After 1 h, the crude product was dissolved in dichloromethane and precipitated into cold methanol for three times. The product was dried in vacuo prior to characterization.

3.2 Synthesis of Poly(Ethylene Glycol)-b-Poly(ε-Caprolactone) (PEG-b-PCL)

PEG-b-PCL diblock copolymers were synthesized according to a previously reported method. (H-J Li et al., Proc. Nat. Acad. Sci., 2016, 113, 4164-4169, incorporated herein by reference). In a typical polymerization, mPEG (weight varies with Mn, 1.0 equiv.) was dissolved in ε-CL (5.0 g, 87.6 equiv.) and stirred for 0.5 h at 100° C. Thereafter, Sn(Oct)2 (17.7 mg, 0.0438 mmol) was introduced to start the polymerization. The reaction mixture was stirred for 12 h at 120° C. under nitrogen atmosphere. After cooling to room temperature, the polymer was dissolved in dichloromethane, precipitated into cold diethyl ether, filtrated, and dried under vacuum.

3.3 Oligonucleotide Synthesis

Oligonucleotides were synthesized on a Model 391 DNA synthesizer (Applied Biosystems, Inc., Foster City, Calif.) using standard solid-phase phosphoramidite methodology. DNA strands were cleaved from the CPG support using ammonium hydroxide (28-30% NH3 basis) at room temperature for 24 h. Dibenzocyclooctyne (DBCO)-terminated DNA was purified by reverse-phase HPLC liquid chromatography. The successful syntheses of all DNA sequences were confirmed by MALDI-ToF MS. The DNA sequences used in this work are shown in Table S1.

3.4 Synthesis of PCL-b-DNA

Azide-terminated PCL (4 mg, 400 nmol) were dissolved in 500 μL of DMSO and DBCO-modified DNA (200 nmol) was dissolved in 50 μL Nanopure™ water. The two solutions were mixed and the reaction mixture was shaken at 45° C. for 24 h, before being dialyzed against NanopureTM water to remove DMSO and precipitate unreacted PCL. The cloudy dialysate was then centrifuged (14,000 rpm for 10 min) to remove solid precipitants. The supernatant was collected and unreacted DNA was removed by reverse-phase HPLC liquid chromatography. Finally, the purified DNA-b-PCL solution was lyophilized to yield a white powder (or colored powder for those containing fluorophore or quencher labels).

3.5 Micellization Procedure

Micelles were prepared by nanoprecipitation.2 Briefly, micelle components, e.g. PEG-b-PCL, DNA-b-PCL, and PCL, were weighed to give a certain molar ratio, and were fully dissolved in DMSO under mixing to give ˜1 mg/mL total concentration. Equal volume of

Nanopure™ water was then added to the mixture using a syringe pump over 2 h. Subsequently, the mixture was dialysed against NanopureTM water to remove the organic solvent.

3.6 Estimation of Surface Moiety Densities

The surface density of oligonucleotides and PEG for each particle was calculated by dividing the number of oligonucleotides per particle by the calculated surface area (cm2).3 The micelle core size is obtained by measuring 500+ particles from non-stained TEM images, as the semi-crystalline PCL cores have more electron density than the shell.4 Several assumptions regarding these calculations were made. First, the DNA-PEG nanoparticles were modeled as perfect spheres. Second, the numbers of particles larger and smaller than the average particle size were assumed to be equal. Third, the oligonucleotides or PEG were assumed to be evenly distributed on the nanoparticle surface. Finally, the PCL core of these nanoparticles were assumed to be solid.

m=ρ×V, ρ=1.07 g/cm3; V=4/3πR3

n=m/Mn×NA, NA=6.02×1023 mol−1

coverage (oligonucleotides or PEG/cm2)=n/S, S=4πR2

m: the mass of the core of DNA-PEG nanoparticles; p: the density of PCL; V: the volume of the core; R: the radius of the core; n: the number of oligonucleotides or PEG per particle; Mn: the number-average molecular weight of PCL; NA: Avogadro constant; S: the surface area of the micelle core.

3.7 Hybridization Kinetics

Micelles with Cy3-labeled DNA were each dissolved in PBS buffer (pH=7.4) at a final concentration of 100 nM. Each sample (1 mL) was transferred to a fluorescence cuvette, to which a complementary dabcyl-labeled DNA strand or a dummy (non-complementary) strand (2 equiv.) was added via 2 μL of PBS solution. The fluorescence of the mixture (ex=550 nm, em=570 nm) was monitored every 3 seconds before mixing and thereafter for 60 min using a Cary Eclipse fluorescence spectrometer. The endpoint is determined by

adding a large excess of complementary dabcyl-DNA to the mixture followed by incubation for an extended period of time (>5 h). The kinetics plots are normalized to the endpoint determined for each sample.

3.8 Nuclease Degradation Kinetics

Micelles (1 μM, with Cy3-labeled DNA) were each mixed with their complementary dabcyl-labeled strand (2 μM) in PBS buffer (pH=7.4). The solutions were gently shaken at room temperature overnight. Thereafter, 100 μL of the samples was withdrawn and diluted to 100 nM with assay buffer (10 mM tris, 2.5 mM MgCl2, and 0.5 mM CaCl2, pH=7.5). Subsequently, DNase I (Sigma-Aldrich) was added to the solution at a final concentration of 0.2 unit/mL, and the fluorescence of the samples was monitored every 3 seconds (ex=550 nm, em=570 nm) for 8 h. The endpoint of each sample was determined by adding a large excess of DNase I (ca. 2 units/mL) to the mixture, and the fluorescence was monitored until no additional increase was observed. The kinetics plots were normalized to the endpoints, and all measurements were repeated three times.

3.9 Cell Culture

SKOV3 cells (a human ovarian cancer cell line) and HeLa cells (human uterine cervix carcinoma) were cultured in DMEM supplied with 10% fetal bovine serum, 1% L-glutamine and 1% antibiotics at 37° C. in a humidified atmosphere containing 5% CO2.

3.10 Flow Cytometry

To study the cellular uptake, SKOV3 or Hela cells were seeded in 12-well plates at 2.0×105 cells per well in 1 mL complete DMEM and cultured for 24 h. Thereafter, Cy3-labeled free DNA, micellar nanoparticles, and Lipofectamine-complexed DNA (1 μM DNA concentration, dispersed in serum-free DMEM medium) were added to different wells and the cells were incubated at 37° C. for 4 h. Cells were then washed with PBS three times and treated with trypsin. PBS (2 mL) was added to each culture well, and the solutions were centrifuged for 5 min at 1000 rpm. The cells were then resuspended in 0.5 mL PBS. All samples were analyzed on a BD FACSCalibur flow cytometer.

3.11 Confocal Laser Scanning Microscope

Cells were seeded in 24-well glass bottom plates at a density of 1.0×105 cells/well and cultured overnight. Serum-free DMEM containing Cy3-labeled free DNA, micellar nanoparticles, and Lipofectamine-complexed DNA were added to each well, followed by incubation for 4 h at 37° C. Thereafter, cells were washed with PBS three times and fixed with 4% paraformaldehyde for 15 min at room temperature. The slides were then rinsed with PBS three times, before being stained with Hoechst 33342 for 10 min. The cells were imaged on an LSM-700 confocal laser scanning microscope (Carl Zeiss Ltd., Cambridge, UK) at excitation wavelengths of 408 nm (Hoechst 33342) and 543 nm (Cy3).

3.12 MTT Assay

Cells were seeded into 96-well plates at an initial density of 1.0×104 cells per well in 200 μL of medium. After culturing for 24 h, cells were treated with free DNA, micellar nanoparticles, and Lipofectamine-complexed DNA at varying concentrations of total DNA (0.05, 0.1, 0.2, 0.5 and 1 μM). Lipofectamine 2000 (Invitrogen) was used under conditions suggested by the manufacturer. The cells were grown in a humidified environment with 5% CO2 at 37° C. for another 48 h. Thereafter, 20 μL of 5 mg/mL MTT assays stock solution in PBS were added to each well. After incubating the cells for 4 h, the medium containing unreacted dye was removed carefully. The obtained blue formazan crystals were dissolved in 200 μL DMSO per well and the absorbance was measured using a microplate reader (Biotek Synergy HT) at a wavelength of 490 nm.

3.13 Western Blotting

Cells were seeded in 6-well plates at a density of 2.0×105 cell per well in 2 mL of DMEM complete medium and allowed to attach for 24 h. Thereafter, cells were treated with free DNA, micellar nanoparticles, and Lipofectamine-complexed DNA at the same DNA concentration (1 μM) for 24 h. The medium was then replaced with fresh, full growth medium and cells were cultured for another 48 h before being harvested. Whole cell lysate was prepared in 100 μL of RIPA Cell Lysis Buffer with 1 mM phenylmethanesulfonylfluoride (PMSF, Cell Signaling Technology) according to the manufacturer's suggested protocol. Protein content in the extracts was quantified using a bicinchoninic acid (BCA) protein assay kit. Equal amounts (30 μg/lane) of protein samples were separated by 4-20% SDS-PAGE and electro-transferred to nitrocellulose membrane. The membranes were then blocked with 3% BSA (bovine serum albumin) in TBST (Tris buffered saline supplemented with 0.05% Tween-20) and further incubated with β-actin (1:1000 dilution) and Bcl-2 (1:1000 dilution) primary and secondary antibodies (Invitrogen). Protein bands were detected by chemiluminescence using the ECL Western blotting substrate (Themo Scientific, USA) according to the manufacturer's protocol.

TABLE 1
Oligonucleotide sequences

Bcl2 antisense strand
5′-DBCO-TTT TCT CCC AGC GTG CGC CAT-3′
(SEQ ID NO: 1)
Cy3-labeled Bcl2 antisense strand
5′-DBCO-TTT TCT CCC AGC GTG CGC CAT-Cy3-3′
(SEQ ID NO: 2)
Dabcyl-labeled Bcl2 sense strand
5′-Dabcyl-ATG GCG CAC GCT GGG AGA AAA-3′
(SEQ ID NO: 3)
Dabcyl-labeled scrambled strand
5′-Dabcyl-ACA CGT TGG GAG GCA GCG AAA-3′
(SEQ ID NO: 4)

TABLE 2
NMR (a) and DMF GPC (b) analyses for the polymers used

| Polymers | Mna (kDa) | Mnb (kDa) | Mwb (kDa) | PDIb |
|---|---|---|---|---|
| PCL-N₃ | 10.5 | 10.2 | 13.1 | 1.28 |
| PEG2k-b-PCL | 11.2 | 10.8 | 14.0 | 1.30 |
| PEG5k-b-PCL | 17.1 | 16.9 | 20.8 | 1.23 |
| PEG10k-b-PCL | 20.1 | 19.7 | 23.1 | 1.17 |

TABLE 3
DLS number-average hydrodynamic diameter (Dh(n)) and zeta potential
measurements

| | PEG- | | | |
|---|---|---|---|---|
| Sample | b-PCL:DNA- | Dh(n) | | |
| potential (mV) | b-PCL:PCL | (nm) | PDI | Zeta |
| Free DNA | n.a. | n.a. | n.a. | −45.8 |
| DNA-b-PCL | n.a. | 35 | 0.157 | −45.5 |
| PEG2k-b-PCL:DNA-b-PCL | 10:1 | 48 | 0.129 | −32.3 |
| PEG5k-b-PCL:DNA-b-PCL | 10:1 | 55 | 0.121 | −20.9 |
| PEG10k-b-PCL:DNA-b-PCL | 10:1 | 64 | 0.057 | −9.24 |
| PEG10k-b-PCL:DNA-b-PCL | 20:1 | 70 | 0.095 | −6.52 |
| PEG10k-b-PCL:DNA-b-PCL:PCL | 20:1:20 | 102 | 0.130 | −7.28 |
| PEG10k-b-PCL:DNA-b-PCL:PCL | 20:1:40 | 120 | 0.125 | −8.15 |
| PEG10k-b-PCL:DNA-b-PCL:PCL | 20:1:80 | 133 | 0.083 | −10.4 |
| PEG10k-b-PCL:DNA-b-PCL:PCL | 20:1:200 | 181 | 0.076 | −17.5 |

TABLE 4
Number-average micelle core size as estimated from uranyl acetate-stained TEM images
and surface moiety density.

| Sample | Diameter (nm) | Oligos/cm2 | PEGs/cm2 |
|---|---|---|---|
| DNA-b-PCL | 27 ± 2 | 2.8 × 10¹³ ± 2.0 × 10¹² | n.a |
| PEG2k-b-PCL:DNA- | 39 ± 3 | 3.8 × 10¹² ± 2.9 × 10¹¹ | 3.8 × 10¹³ ± 2.9 × 10¹² |
| b-PCL | | | |
| (10:1) | | | |
| PEG5k-b-PCL:DNA- | 47 ± 2 | 4.6 × 10¹² ± 2.0 × 10¹¹ | 4.6 × 10¹³ ± 2.0 × 10¹² |
| b-PCL | | | |
| (10:1) | | | |
| PEG10k-b-PCL:DNA- | 49 ± 3 | 4.8 × 10¹² ± 2.9 × 10¹¹ | 4.8 × 10¹³ ± 2.9 × 10¹² |
| b-PCL | | | |
| (10:1) | | | |
| PEG10k-b-PCL:DNA- | 62 ± 3 | 3.2 × 10¹² ± 1.5 × 10¹¹ | 6.3 × 10¹³ ± 3.1 × 10¹² |
| b-PCL | | | |
| (20:1) | | | |
| PEG10k-b-PCL:DNA- | 96 ± 5 | 2.5 × 10¹² ± 1.3 × 10¹¹ | 5.0 × 10¹³ ± 2.6 × 10¹² |
| b-PCL:PCL | | | |
| (20:1:20) | | | |
| PEG10k-b-PCL:DNA- | 112 ± 6 | 2.0 × 10¹² ± 1.1 × 10¹¹ | 3.9 × 10¹³ ± 2.1 × 10¹² |
| b-PCL:PCL | | | |
| (20:1:40) | | | |
| PEG10k-b-PCL:DNA- | 124 ± 4 | 1.3 × 10¹² ± 4.3 × 10¹⁰ | 2.6 × 10¹³ ± 8.5 × 10¹¹ |
| b-PCL:PCL | | | |
| (20:1:80) | | | |
| PEG10k-b-PCL:DNA- | 155 ± 5 | 7.5 × 10¹¹ ± 2.4 × 10¹⁰ | 1.5 × 10¹³ ± 4.9 × 10¹¹ |
| b-PCL:PCL | | | |
| (20:1:200) | | | |

TABLE 5
DNA half-lives (100 nM DNA) in the presence of 0.2 unit/mL DNase I.

| | Half-lives | | Half-lives |
|---|---|---|---|
| Sample | (min) | Sample | (min) |
| Free DNA | 12 ± 0.5 | PEG10k-b-PCL:DNA-b- | 240 ± 18 |
| | | PCL (20:1) | |
| DNA-b-PCL | 36 ± 1 | PEG10k-b-PCL:DNA-b- | 238 ± 20 |
| | | PCL:PCL (20:1:20) | |
| PEG2k-b-PCL:DNA- | 22 ± 2 | PEG10k-b-PCL:DNA-b- | 120 ± 16 |
| b-PCL | | PCL:PCL (20:1:40) | |
| (10:1) | | | |
| PEG5k-b-PCL:DNA- | 43 ± 5 | PEG10k-b-PCL:DNA-b- | 67 ± 5 |
| b-PCL | | PCL:PCL (20:1:80) | |
| (10:1) | | | |
| PEG10k-b-PCL:DNA- | 142 ± 15 | PEG10k-b-PCL:DNA-b- | 40 ± 4 |
| b-PCL | | PCL:PCL (20:1:200) | |
| (10:1) | | | |

### Example 2

### Relationship of Micelle Structural Parameters and Steric Selectivity

To systematically probe the relationship between the structural parameters of

DNA-PEG nanoparticles and their steric selectivity, two series of nanostructures are prepared: 1) micelles containing DNA-b-PCL and PEG-b-PCL copolymers having varying PEG lengths, and 2) micelles containing DNA-b-PCL, PEG₁₀ₖ-b-PCL, and varying amounts of PCL homopolymer. The first series emphasizes the effect of the relative lengths of the PEG and the DNA, while the second series enables the study of the surface PEG density.

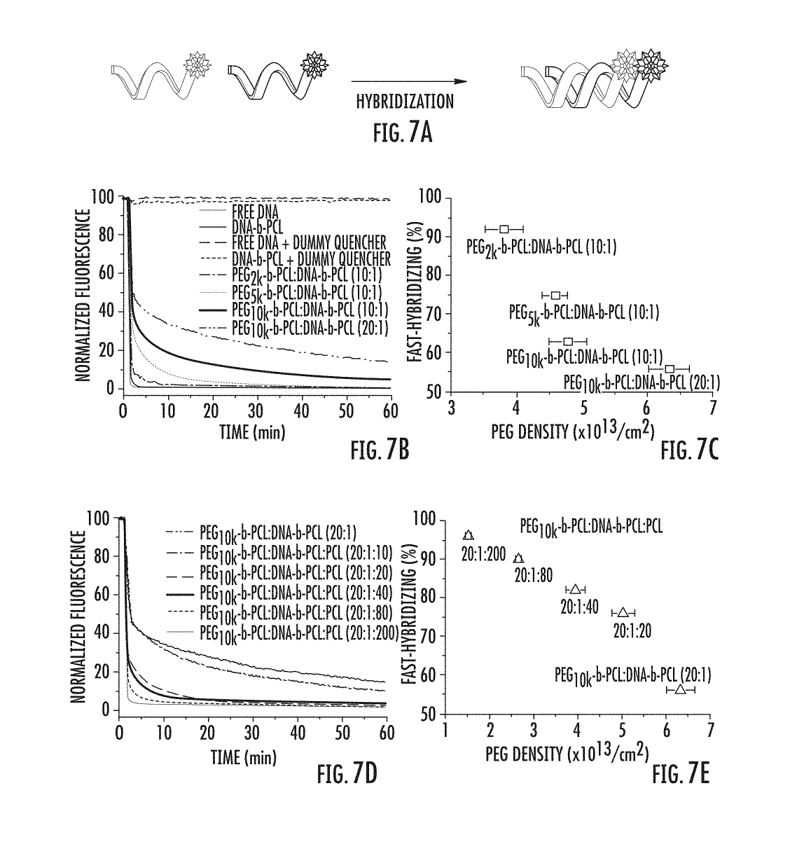

To study the ability of the oligonucleotide (DNA) component within the co-assembled micelles to hybridize with a complementary (sense) sequence, a fluorescence quenching assay was used (Lu, X et al., J. Am. Chem. Soc. 2015, 137, 12466-12469), in which a quencher (dabcyl)-modified sense strand is mixed with Cy3-labeled particles. Upon hybridization, the fluorophore-quencher pair is brought to proximity, leading to a reduction in the fluorescence signals (FIG. 7A). The rate of fluorescence reduction is therefore an indicator of the hybridization kinetics. A scrambled strand is used as a control to rule out the possibility of non-specific interactions. As shown in FIG. 7B both free DNA and micellar nanoparticles can hybridize with the sense strand, while the scrambled sequence causes no change in the fluorescent signals. The micelles exhibit a two-population behavior: a population with rapid hybridization kinetics similar to that of free DNA, and a slow-hybridizing population, which increases with increasing PEG content (FIGS. 7B and 7C). Notably, micelles containing no PEG-b-PCL (spherical nucleic acid-like micelles), comprise almost entirely the fast-hybridization population. The slow-hybridizing population is attributed to the sub-micellar microstructure, which causes excessive hindrance around the DNA and is affected by the density of the PEG. By diluting the micelle core with PCL homopolymer (thus a decrease in surface PEG density), the fast-hybridizing population can be almost fully restored (FIGS. 7D and 7E).

### Example 3

### Micelle Structure and Enzyme Accessibility

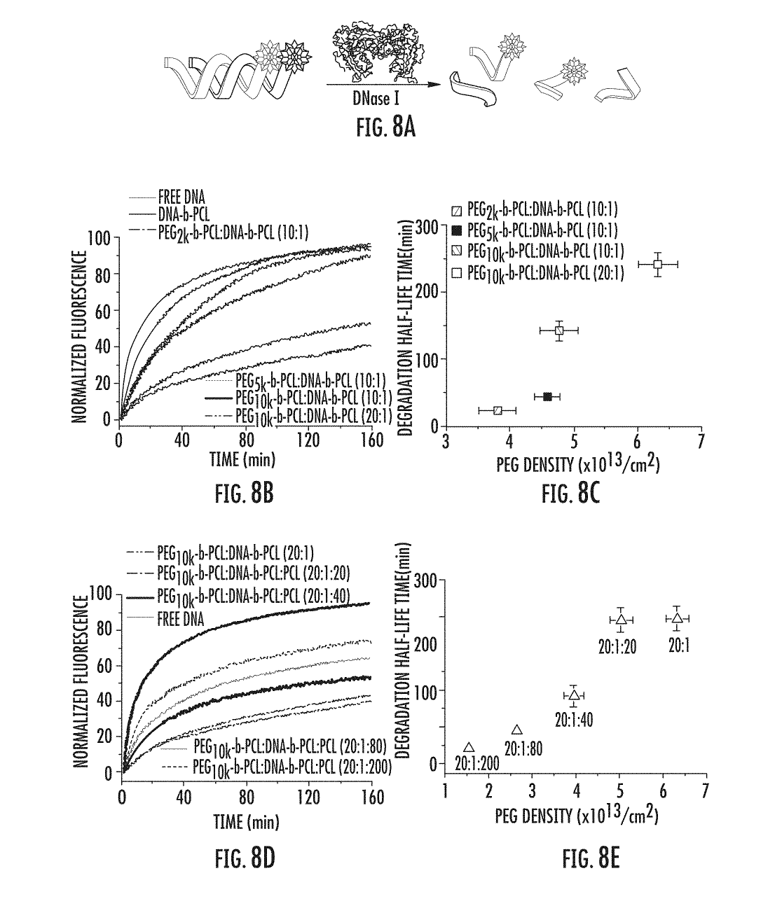

The enzyme accessibility of the oligonucleotide component within the micelles was analyzed, using bovine pancreas DNase I as a model enzyme. Cy3-labeled micelles are pre-hybridized with quencher-labeled sense strands. Upon introduction of DNase I, the dsDNA is degraded, resulting in an increase in fluorescence (FIG. 8A). As shown in FIG. 8B, naked dsDNA is readily accessed and degraded in the presence DNase I, with a half-life (t0.5) of 12.0±0.5 min. In contrast, micelles consisting of pure DNA-b-PCL show slightly enhanced nuclease resistance (t0.5: 36±1 min) due to increased steric hindrance and local high salt concentrations, which is consistent with previous reports. Adding PEG2k-b-PCL to the micelle (10:1 m:m PEG:DNA amphiphile ratio) results in an increase in degradation rate (t0.5: 22±2 min). Amphiphiles of higher PEG molecular weight (5 and 10 kDa), on the other hand, enhance DNA stability under identical conditions. The highest stability is achieved with PEG10k-b-PCL, which gives a t0.5 of 142±15 min at 10:1 PEG:DNA amphiphile ratio, and 240±18 min at 20:1. One interpretation of these results is that longer PEG chains can more effectively shield the underlying DNA strands from enzymatic access (FIG. 8C and Table 3). When the PEG block is too short, the diluting effect of the PCL block becomes dominant, which serves to increase the spacing of DNA strands, leading to more facile enzyme access and degradation.

A systematic investigation as to how a homopolymer, e.g., PCL, in the nanoparticle formulation can be used to tune the spacing of particle surface moieties and maximize DNA binding selectivity was conducted. Micelles consisting of PEG10k-b-PCL and DNA-b-PCL (20:1 m:m) are used as a base composition, to which different molar ratios of PCL (10-200, relative to DNA-b-PCL) are added. It is found that PCL significantly increases particle core size, from 62±2 nm (base composition, estimated from TEM images) to 155±5 nm (with 200 equiv. PCL), which correlates with a drop of surface PEG density from 6.3 ×1013 PEG/cm2 to 1.5×1013 PEG/cm2 (see Table 4). The reduction in PEG density results in acceleration in both hybridization and enzymatic access, with the change in hybridization being faster. With 20 equiv. of PCL (density: 5.0×1013 PEG/cm2), hybridization can be mostly restored (>85% hybridized in 5 min), while protein shielding remains nearly unaffected (t0.5 of 238 min vs. 240 min for base composition, FIGS. 8D and 8E). In contrast, excessive of PCL (200 equiv.) results in incremental improvements in hybridization, but substantial loss in enzyme stability, showing a t0.5 of ˜40 min. These results demonstrate that PCL homopolymer can be used to tune the PEG density of the micelles, thereby opening a window in which DNA binding selectivity can be maximized.

### Example 4

### Micelle Structure and Cellular Uptake

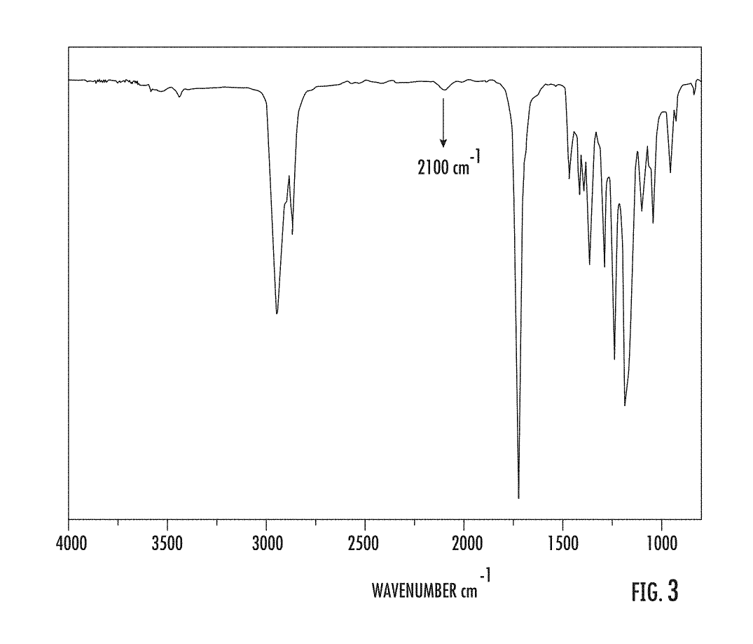

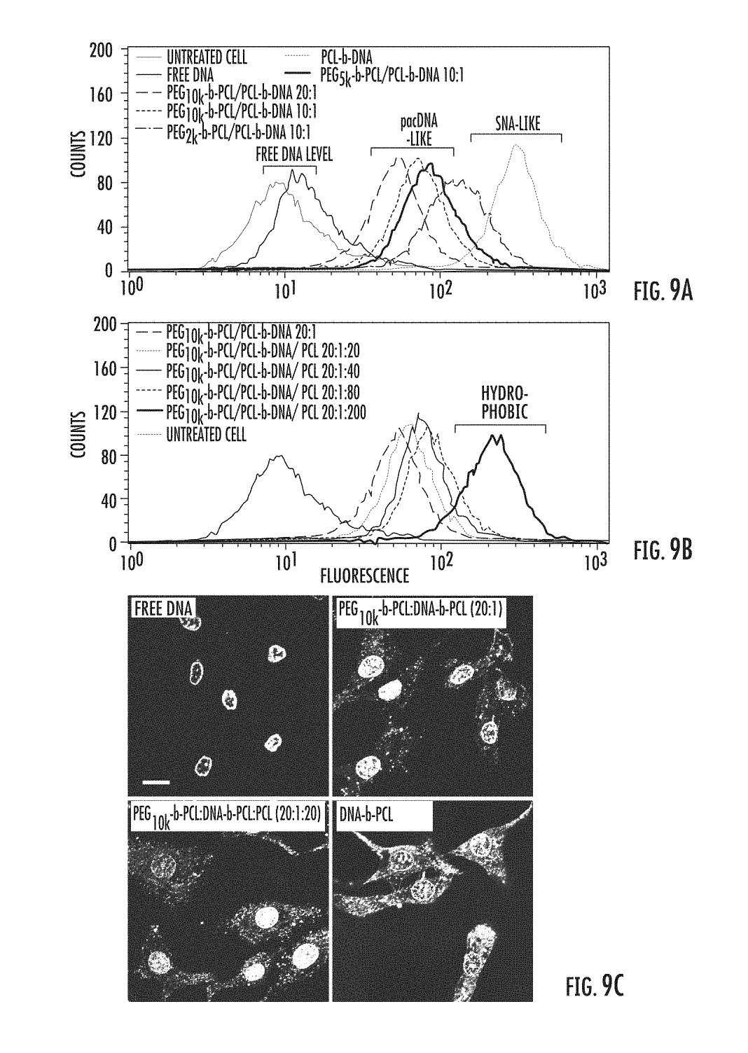

In addition to nuclease stability, efficient cellular uptake is another important aspect in high antisense efficacy, for example. To examine the extent of endocytosis, SKOV3 cells are incubated with a library of Cy3-labeled micelles and free DNA having the same DNA concentration (1 μM) for 4 h. Flow cytometry shows that, compared with free DNA, co-assembled micelles have enhanced cell uptake (20-90× relative to free DNA, FIG. 3), with shorter PEG and lower PEG densities leading to more uptake (FIG. 9A). Micelles with no surface PEG (DNA-b-PCL only) show the highest uptake at ˜150× that of free DNA. These PEG-free micelles are structurally analogous to spherical nucleic acids (SNAs), which have been shown to exhibit high, non-specific cell uptake due to recognition by class A scavenger receptors and endocytosis via a lipid-raft-dependent, caveolae-mediated path way. With the addition of moderate amounts of PCL homopolymer in the micellar core, cellular uptake is increased, but only to a small extent (FIG. 9B). However, when 200 equiv. of PCL is added, cell uptake is augmented to SNA-like levels. The unusually high cellular uptake is likely associated with the exposed hydrophobic regions of the micelle surface caused by excessive loading of PCL, which interacts with cells in a different mechanism than PEG- and DNA-dominated surfaces. These results are corroborated by confocal laser scanning microscopy. While cells treated with free DNA show no or very weak fluorescence, DNA micelles give much stronger signals under the identical imaging settings, with increasing PEG contents reducing the intensities of fluorescent signals (FIG. 9C). The same experiments were also performed with Hela cells, which show a similar general trend. Collectively, the results indicate that the uptake of DNA-PEG-PCL micelles can be tuned by adjusting the surface composition exposed to the cell, from being PEG-like to SNA-like, and to hydrophobic.

### Example 5

### Relationship of Micelle Structural Parameters and Gene Silencing Efficacy

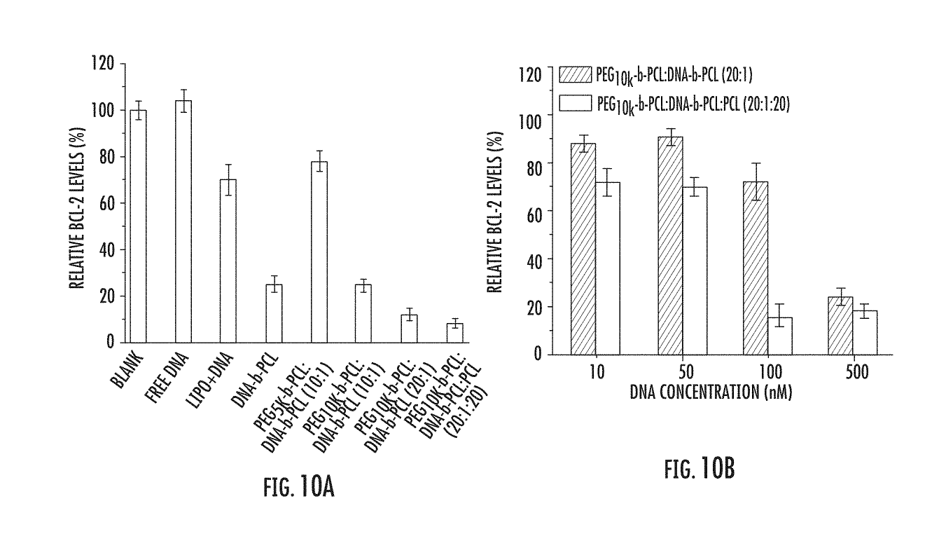

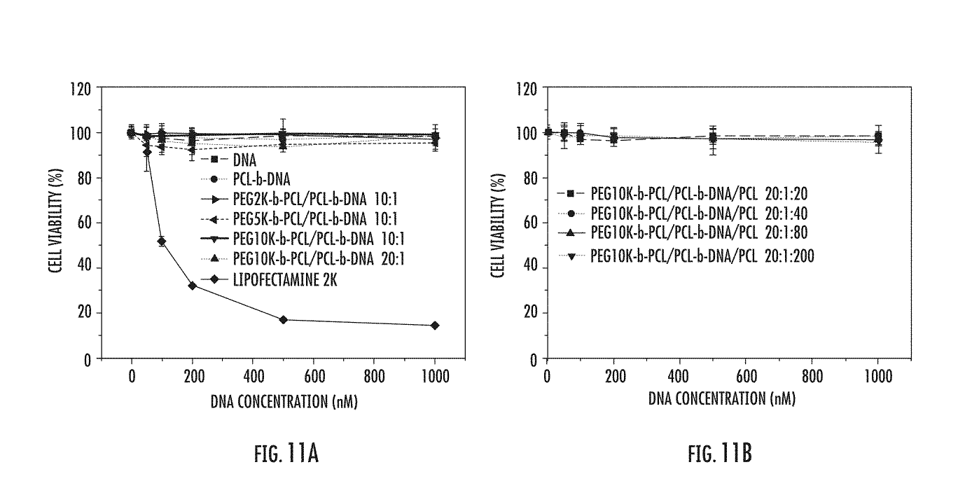

How different micelle structures affect antisense gene silencing efficacy was analyzed. Because cellular endosomes are associated with digestive environments that can degrade the antisense sequence, higher stability may contribute to greater overall efficacy. SKOV3 cells were treated with the two series of micelles described above at an equal dose of DNA (1 μM) for 24 h, followed by culturing for 48 h in fresh media. Lipofectamine-complexed DNA and free DNA are used as positive and negative controls, respectively. The levels of Bcl-2 were analyzed by western blotting. As shown in FIG. 10A and FIG. 11A the Bcl-2 levels are significantly reduced by micelles having 10 kDa PEG amphiphiles. The best of these is PEG10k-b-PCL:DNA-b-PCL:PCL (20:1:20), showing 92% reduction in expression (band densitometry analysis, normalized to β-actin). These micelles coincide with those showing the highest nuclease stability, but moderate cellular uptake and hybridization readiness, suggesting that protein inhibition is a key factor in achieving high efficacy. Indeed, low-stability micelles having 5 kDa PEG show considerably lower efficacy (22% knockdown for PEG5k-b-PCL:DNA-b-PCL, 10:1), even though they exhibit better cell uptake and hybridization. Although pure DNA-b-PCL micelle (SNA-like) only shows a small level of nuclease resistance, its antisense activity is significant (75% knock-down). This phenomenon is attributed to the unusually high cellular uptake associated with the SNAs.

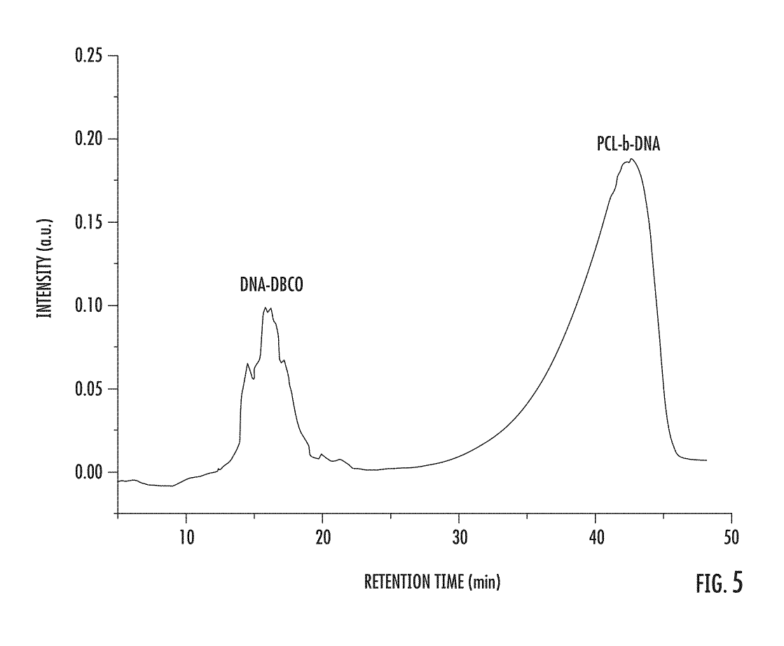

A dose-dependent study for the two-component system, PEG10k-b-PCL:DNA-b-PCL (20:1), and the corresponding three-component system with 20 equiv. of PCL was also performed. It is found that both systems are effective at high concentrations (>500 nM), but at a lower concentration (100 nM), the three-component system shows higher gene knock down efficacy (84% vs. 18%, FIGS. 5B and 7), signifying that increased hybridization availability (FIG. 7D) of the antisense ON is important, since these two systems show roughly the same nuclease stability. Because the tested micelles consist primarily of DNA, PEG, and PCL, components regarded by the US Food and Drug Administration as generally safe for pharmaceuticals, it is anticipated that these co-assembled nanoparticles are non-cytotoxic. Indeed, a 3-(4,5-dimethyl-thiazol-2-yl)-2,5-diphenyl tetrazolium bromide (MTT) assay of SKOV3 cells treated with the micelles shows that viability after 48 h of incubation remains nearly 100%. In contrast, DNA complexed with Lipofectamine exhibit significant cytotoxicity (>50% cell death at 100 nM of DNA), as expected from typical polycationic carrier systems (FIG. 11B).

1. An oligonucleotide-loaded amphiphilic micelle, said micelle being co-assembled from (1) a first amphiphilic polymer comprising a hydrophobic polymer covalently coupled to an oligonucleotide; (2) a second amphiphilic polymer; and (3) an optional hydrophobic homopolymer or hydrophobic small molecule, wherein said micelle has an outer surface density of hydrophilic polymer in the range of from 1×10¹² to 3×10¹⁴ hydrophilic polymer/cm².

2. The oligonucleotide-loaded amphiphilic micelle of claim 1, wherein the micelle comprises oligonucleotide-b-poly(ε-caprolactone) (oligo-b-PCL); polyethylene glycol-b-PCL (PEG-b-PCL) and PCL homopolymer.

3. The oligonucleotide-loaded amphiphilic micelle of claim 1, wherein the oligonucleotide is 2 to 50 bases in length.

4. The oligonucleotide-loaded amphiphilic micelle of claim 1, wherein the oligonucleotide is siRNA.

5. The oligonucleotide-loaded amphiphilic micelle of claim 1, wherein the oligonucleotide is single stranded or double stranded RNA or DNA.

6. The oligonucleotide-loaded amphiphilic micelle, wherein the oligonucleotide is labelled with a detectable marker.

7. The oligonucleotide-loaded amphiphilic micelle of claim 2, wherein the PEG has a molecular weight of from 2 to 50 kDa.

8. The oligonucleotide-loaded amphiphilic micelle of claim 1, wherein the density of hydrophilic polymer at the surface of the micelle is from 1×10¹² to 3×10¹⁴ PEG/cm² micelle.

9. An amphiphilic peptide-loaded micelle, said micelle being co-assembled from (1) a first amphiphilic polymer comprising a hydrophobic polymer coupled to a peptide; (2) a second amphiphilic polymer; and (3) an optional hydrophobic homopolymer or hydrophobic small molecule, wherein said micelle has an outer surface density of hydrophilic polymer in the range of from 1×10¹² to 3×10¹⁴ hydrophilic polymer/cm².

10. The amphiphilic peptide-loaded micelle of claim 1, wherein the peptide is 2 to 100 amino acids in length.

11. The amphiphilic peptide-loaded micelle of claim 9, wherein the micelle comprises peptide-b-poly(ε-caprolactone) (peptide-b-PCL); polyethylene glycol-b-PCL (PEG-b-PCL) and PCL homopolymer.

12. The amphiphilic peptide-loaded micelle of claim 11, wherein, wherein the PEG has a molecular weight of from 2 to 50 kDa.

13. The amphiphilic peptide-loaded micelle of claim 11, wherein, wherein the density of PEG at the surface of the micelle is from 1×10¹² to 3×10¹⁴ PEG/cm² micelle.

14. A small molecule-loaded amphiphilic micelle, said micelle being co-assembled from (1) an amphiphilic polymer; (2) a small molecule; and optionally, (3) a hydrophobic homopolymer, wherein said small molecule is associated with the micelle through non-covalent interactions, and wherein said micelle has an outer surface density of hydrophilic polymer in the range of from 1×10¹² to 3×10¹⁴ hydrophilic polymer/cm².

15. A method for promoting cellular uptake of an oligonucleotide or peptide in a subject or biological sample, said method comprising delivering an oligonucleotide-loaded or peptide-loaded micelle to the subject or contacting the biological sample with the oligonucleotide-loaded or peptide-loaded micelle, respectively, wherein the oligonucleotide-loaded or peptide-loaded micelle comprises (1) a first amphiphilic polymer comprising an oligonucleotide coupled to a hydrophobic polymer or a peptide coupled to a hydrophobic polymer; (2) a second amphiphilic polymer; and (3) an optional hydrophobic homopolymer, wherein said micelle has an outer surface density of hydrophilic polymer in the range of from 1×10¹² to 3×10¹⁴ hydrophilic polymer/cm².

16. A method of detecting the presence of a target polynucleotide in a subject or a tissue sample obtained from a subject, said method comprising contacting the target polynucleotide with an oligonucleotide-loaded micelle, said oligonucleotide-loaded micelle being co-assembled from (1) a first amphiphilic polymer covalently coupled to the oligonucleotide; (2) a second amphiphilic polymer polymer; and (3) an optional hydrophobic homopolymer or hydrophobic small molecule, wherein said micelle has an outer surface density of hydrophilic polymer in the range of from 1×10¹² to 3×10¹⁴ hydrophilic polymer/cm² and wherein the oligonucleotide is complementary to and hybridizes to at least a portion of the target polynucleotide.

17. A method for inhibiting expression of a gene product encoded by a target polynucleotide, said method comprising contacting a target polynucleotide with an amphiphilic micelle, said micelle being co-assembled from (1) a first amphiphilic polymer comprising a hydrophobic polymer covalently coupled to an oligonucleotide; (2) a second amphiphilic polymer polymer; and (3) an optional hydrophobic homopolymer or hydrophobic small molecule, wherein said micelle has an outer surface density of hydrophilic polymer in the range of from 1×10¹² to 3×10¹⁴ hydrophilic polymer/cm² and wherein the oligonucleotide is complimentary to and hybridizes to at least a portion of the target polynucleotide.

18. The method of claim 17, wherein the oligonucleotide is siRNA.

19. The micelle of claim 1, wherein the first and second amphiphilic polymers and the hydrophobic homopolymer are biocompatible and biodegradable.

20. The micelle of claim 1, wherein the oligonucleotide is coupled to the polymer via a cleavable bond.


SEQUENCE LISTING (4 sequences)

SEQ ID NO: 1 - DNA, 21 bp, Artificial Sequence
  Description of Artificial Sequence Synthetic oligonucleotide
ttttctcccagcgtgcgccat

SEQ ID NO: 2 - DNA, 21 bp, Artificial Sequence
  Description of Artificial Sequence Synthetic oligonucleotide
ttttctcccagcgtgcgccat

SEQ ID NO: 3 - DNA, 21 bp, Artificial Sequence
  Description of Artificial Sequence Synthetic oligonucleotide
atggcgcacgctgggagaaaa

SEQ ID NO: 4 - DNA, 21 bp, Artificial Sequence
  Description of Artificial Sequence Synthetic oligonucleotide
acacgttgggaggcagcgaaa